SEQUENCE LISTING (22 sequences)

SEQ ID NO: 1 - protein, 10 aa, Artificial Sequence
  Description of Artificial Sequence Synthetic peptide
WWRRRRRRRR

SEQ ID NO: 2 - protein, 8 aa, Artificial Sequence
  Description of Artificial Sequence Synthetic peptide
RRRRRRRR

SEQ ID NO: 3 - protein, 9 aa, Mus sp.
FALMNRKAL

SEQ ID NO: 4 - protein, 9 aa, Mus sp.
MAPIDHTAM

SEQ ID NO: 5 - protein, 9 aa, Mus sp.
SAIRSYQDV

SEQ ID NO: 6 - protein, 9 aa, Mus sp.
KSFHFYCRL

SEQ ID NO: 7 - protein, 9 aa, Mus sp.
LSAARYALL

SEQ ID NO: 8 - protein, 9 aa, Mus sp.
ASYSQVAHI

SEQ ID NO: 9 - protein, 9 aa, Mus sp.
CGYEHTAVL

SEQ ID NO: 10 - protein, 9 aa, Mus sp.
MSYFLQGKL

SEQ ID NO: 11 - protein, 9 aa, Mus sp.
AQLPNDVVL

SEQ ID NO: 12 - protein, 9 aa, Mus sp.
ASMTNRELM

SEQ ID NO: 13 - protein, 9 aa, Artificial Sequence
  Description of Artificial Sequence Synthetic peptide
FALMNLKAL

SEQ ID NO: 14 - protein, 9 aa, Artificial Sequence
  Description of Artificial Sequence Synthetic peptide
MAPIDHTTM

SEQ ID NO: 15 - protein, 9 aa, Artificial Sequence
  Description of Artificial Sequence Synthetic peptide
SAIRSYQYV

SEQ ID NO: 16 - protein, 9 aa, Artificial Sequence
  Description of Artificial Sequence Synthetic peptide
KSFHFYCPL

SEQ ID NO: 17 - protein, 9 aa, Artificial Sequence
  Description of Artificial Sequence Synthetic peptide
LSASRYALL

SEQ ID NO: 18 - protein, 9 aa, Artificial Sequence
  Description of Artificial Sequence Synthetic peptide
ASYSLVAHI

SEQ ID NO: 19 - protein, 9 aa, Artificial Sequence
  Description of Artificial Sequence Synthetic peptide
CVYEHTAVL

SEQ ID NO: 20 - protein, 9 aa, Artificial Sequence
  Description of Artificial Sequence Synthetic peptide
MSYFLQGTL

SEQ ID NO: 21 - protein, 9 aa, Artificial Sequence
  Description of Artificial Sequence Synthetic peptide
AQLANDVVL

SEQ ID NO: 22 - protein, 9 aa, Artificial Sequence
  Description of Artificial Sequence Synthetic peptide
ASMTNMELM

(19) United States
(12) Patent Application Publication
(10) Pub. No.: US 2020/0224206 A1
(43) Pub. Date: Jul. 16, 2020

(54) Amplicon Expression Vector Vaccines

(71) Applicants: LineaRx, Inc., Stony Brook, NY (US); Evvivax S. R. L., Roma (IT)

(72) Inventors: Michael E. Hogan, Stony Brook, NY (US); Stephen Hughes, Port Jefferson Station, NY (US); Antonella Conforti, Roma (IT); Luigi Aurisicchio, Roma (IT); Fabio Palombo, Roma (IT)

(21) Appl. No.: 16/703,915

(22) Filed: Dec. 5, 2019

Related U.S. Application Data
(60) Provisional application No. 62/775,604, filed on Dec. 5, 2018.

(51) Int. Cl. C12N 15/64; A61K 38/17; A61P 35/00; A61K 41/00; A61K 39/00

(52) U.S. Cl. CPC C12N 15/64; A61K 38/1774; A61P 35/00; A61K 2039/54; A61K 39/00116; A61K 39/001157; A61K 2039/53; A61K 41/0047

(57) ABSTRACT

Provided herein are non-plasmid derived DNA vaccines comprised solely of enzymatically produced amplicon expression vectors and their method of use to elicit antigen-specific immune responses in a subject. The enzymatically produced amplicon expression vectors may be specifically utilized as a DNA based cancer vaccine to express desired antigens or other immunogenic polypeptides within a subject to induce a specific anti-cancer antigen-specific immune response. The enzymatically produced amplicon expression vectors may also be utilized to express cancer-specific neoantigens.

### CROSS-REFERENCE TO PROVISIONAL PATENT APPLICATION AND JOINT DEVELOPMENT

The present application claims the benefit of U.S. Provisional Patent Application Ser. No. 62/775,604 filed Dec. 5, 2018. In addition, the present application relates to the subject of a joint development agreement by and between co-applicants LineaRx, Inc., a subsidiary of Applied DNA Sciences, Inc. and Evvivax S.R.L.

### TECHNICAL FIELD

Provided herein are DNA vaccines comprised of enzymatically produced amplicon expression vectors and their methods of use to elicit antigen-specific immune responses in a subject. The enzymatically produced amplicon expression vectors may be specifically utilized as a DNA cancer vaccine to express encoded antigens or other immunogenic polypeptides within a subject's cells to elicit a specific anti-cancer antigen-specific immune response. The enzymatically produced amplicon expression vectors may also be utilized to express cancer-specific neoantigens.

### BACKGROUND

Cancer is the leading cause of mortality worldwide. Conventional therapies such as surgery, radiation and chemotherapy are highly invasive without offering lifelong protection. Recently, immunotherapy has emerged as a promising form of cancer treatment. New immunotherapeutic approaches to cancer treatment have resulted in positive clinical outcomes for patients that just a few years ago would have been impossible.

DNA-based cancer vaccines are an important tool in the cancer immunotherapy toolbox. DNA vaccination represents a simple, safe and promising strategy for harnessing the immune system to combat numerous disparate forms of cancer. DNA vaccines for cancer immunotherapy are designed to deliver one or several genes encoding tumor antigens or other immunogenic polypeptides to modulate immune responses, thereby eliciting or augmenting immune responses against tumor antigens that play a central role in tumor initiation, progression and metastasis, or arise as a result of mutational burden. DNA cancer vaccines can induce both innate immunity activation and adaptive immune responses which can suppress tumor growth and, in some cases, achieve total tumor rejection or destruction.

Heretofore, DNA based vaccines, including DNA cancer vaccines, have involved the inoculation of a subject with plasmid DNA and/or plasmid derived DNA. Bacterial plasmids are episomal circular DNA constructs propagated in bacteria via a fermentation process. The manufacture of DNA vaccines via plasmids has several drawbacks, including without limitation, the presence of antibiotic resistance genes, long lead times, the presence of large amounts of extraneous DNA unrelated to the expression cassette, impurities from bacterial cultures, endotoxins, inefficient uptake of the large plasmid DNA molecules to the cellular nucleus, recombination events, and challenges of integrating plasmid production into automated GMP workflows. Thus, there is an unmet need for a new manner of DNA vaccines that do not utilize an expression vector comprised of, or based upon, bacterial plasmids or plasmid derived DNA.

The manufacture of a DNA vaccine comprised of one or more DNA amplicon expression vectors, manufactured via the polymerase chain reaction (PCR), or other non-plasmid enzymatic amplification technology, eliminates the numerous risks and drawbacks associated with plasmid DNA based vaccines. In addition, through the incorporation of chemical and/or peptide modifications, PCR-produced amplicon expression vectors can be optimized for high-level expression within target cells leading to an enhanced antigen-specific immune response. Furthermore, the use of amplicon expression vectors for DNA cancer vaccines provides the enhanced ability to rapidly manufacture tumor specific cancer vaccines that can elicit antigen-specific immune responses to one or more tumor neoantigens for increased efficacy and reduced on-target of-tumor effects.

### SUMMARY OF INVENTION

Provided herein are DNA vaccines comprised of polymerase chain reaction (PCR) produced amplicon expression vectors and their method of use to elicit antigen-specific immune responses in a subject. The subject may be human or non-human. There are also methods provided herein for the manufacture of PCR produced amplicon expression vectors and methods of non-invasive vaccination with said PCR produced amplicon expression vectors via injection and electroporation of amplicon expression vectors into a subject to produce a desired antigen-specific immune response. The antigen expression within the subject's cells may be any antigen and/or other immunogenic polypeptide capable of eliciting an immune response in the subject via humoral and/or cellular adaptive immunity. The antigens may be associated with any tumor antigen, including without limitation, tumor-specific antigens (TSA), tumor associated antigens (TAA), cancer testis antigens (CTAs), tumor neoantigens, mutational associated neoantigens, neoepitopes and/or oncoviral, oncofetal, lineage-restricted, and/or over-expressed tumor antigens. The antigen and/or other immunogenic polypeptide may also be associated with any infectious diseases derived from an autoimmune condition, allergy, immune deficiency condition, virus, bacteria, fungi or parasite.

Also provided herein is a composition of PCR produced amplicon expression vectors optimized to enhance immunogenicity and efficacy in a subject through chemical, peptide and/or sequence modifications. A composition comprising an amplicon expression vector encoding for one or more desired antigens, wherein said amplicon is peptide modified is disclosed. The amplicon expression vectors may, optionally, contain a fusion open reading frame (ORF) and/or a complexed transfection agent selected from the group of cationic polymers, branched dendrimers and cationic liposomes. The peptide modification to the amplicon expression vectors may comprise one or more cell penetrating peptides (CPP) and/or one or more nuclear localization signal (NLS) containing peptides. The one or more CPP and/or the one or more NLS containing peptides may be covalently or non-covalently bonded to 3′ and/or 5′ termini of the amplicon expression vectors. Moieties conferring protection against endonuclease degradation may also be included. Also provided herein are methods of large-scale PCR-based production of DNA vaccines comprised of amplicon expression vectors via batch-based or continuous flow PCR methodologies.

In one embodiment, a method of treating cancer in a subject is disclosed, said method comprising: (a) identifying in a subject cancer neoantigens; (b) without the use of plasmid-derived DNA, assembling an amplicon expression vector template comprising a promotor, one or more open reading frames (ORF) encoding the identified neoantigens and a terminator via gene synthesis; (c) amplifying the amplicon expression vector template via the polymerase chain reaction (PCR) and phosphorothioate modified PCR primers to produce a plurality of amplicon expression vectors; (d) purifying and concentrating the amplicon expression vectors to create an efficacious therapeutic dose for the subject; and (e) administering the therapeutic dose of amplicon expression vectors via electroporation in conjunction with one or more immune checkpoint inhibitors.

In another aspect of the invention, a method of treating cancer in a subject is disclosed, said method comprising: A method of prophylactically treating cancer in a subject, said method comprising: (a) without the use of plasmid-derived DNA, assembling an amplicon expression vector template comprising a promotor, an ORF encoding a consensus sequence for telomerase reverse transcriptase and terminator; (b) amplifying the amplicon expression vector template via the polymerase chain reaction (PCR) and phosphorothioate modified PCR primers to produce a plurality of amplicon expression vectors; (c) purifying and concentrating the amplicon expression vectors to create an efficacious prophylactic dose for the subject; and (d) administering the prophylactic dose of amplicon expression vectors via electroporation.

In yet another aspect of the invention, a method of treating cancer in a subject is disclosed, said method comprising: (a) without the use of plasmid-derived DNA, assembling an amplicon expression vector template comprising a promotor, an ORF encoding a consensus sequence for telomerase reverse transcriptase and terminator; (b) amplifying the amplicon expression vector template via the polymerase chain reaction (PCR) and phosphorothioate modified PCR primers to produce a plurality of amplicon expression vectors; (c) purifying and concentrating the amplicon expression vectors to create an efficacious therapeutic dose for the subject; (d) and administering the therapeutic dose of amplicon expression vectors via electroporation in conjunction with one or more immune checkpoint inhibitors.

### BRIEF DESCRIPTION OF THE FIGURES

FIG. 1 is a flow diagram of one embodiment for the assembly of amplicon expression vectors.

FIG. 2 is a flow diagram of an alternative embodiment for the assembly of amplicon expression vectors.

FIGS. 3A and 3B Top: Hela cells (10000 cells/well) were treated with equimolar amounts of plasmid or amplicon expression vector in Optimem, 10% FCS. 24 hrs later, luciferin was added in the plate and light emission measured via a Xenogen IVIS 200 system. Bottom: Quantification of luciferase expression. Photons emitted were measured through Living Image software. The blue and orange histograms indicate the expression level efficiency at 1 and 0.3 μg DNA plasmid and equimolar amount of amplicon expression vectors, respectively.

FIGS. 4A and 4B Top: Groups of BALB/c mice were treated with equimolar amounts plasmid or amplicon expression vector via DNA electroporation. 24 hrs later, luciferin was injected i.p. and light emission measured via a Xenogen IVIS 200 system. Only the group 50 μg DNA plasmid and 18,75 μg amplicon expression vectors are shown. Bottom: Quantification of luciferase expression. Photons emitted at the indicated DNA concentrations were measured through Living Image software. The histograms indicate the expression level efficiency of DNA plasmid and equimolar amount of amplicon expression vectors, respectively.

FIGS. 5A and 5B Top: Groups of BALB/c mice were treated with equimolar amounts plasmid or amplicon expression vector via DNA electroporation. 48 hrs later, luciferin was injected i.p. and light emission measured via a Xenogen IVIS 200 system. Only the group 50 μg DNA plasmid and 18,75 μg amplicon expression vectors are shown. Bottom: Quantification of luciferase expression. Photons emitted at the indicated DNA concentrations were measured through Living Image software. The histograms indicate the expression level efficiency of DNA plasmid and equimolar amount of amplicon expression vectors, respectively.

FIGS. 6A and 6B Top: Groups of BALB/c mice were treated with equimolar amounts plasmid or amplicon expression vector via DNA electroporation. 1 week later, luciferin was injected i.p. and light emission measured via a Xenogen IVIS 200 system. Only the group 50 μg DNA plasmid and 18,75 m amplicon expression vectors are shown. Bottom: Quantification of luciferase expression. Photons emitted at the indicated DNA concentrations were measured through Living Image software. The histograms indicate the expression level efficiency of DNA plasmid and equimolar amount of amplicon expression vectors, respectively.

FIG. 7 Groups of BALB/c mice were treated with equimolar amounts plasmid or amplicon expression vector encoding canine telomerase reverse transcriptase (dTERT) via DNA electroporation at days 0 and 21. 1 week later on day 28, mice were bled and PBMC prepared and stimulated with antigen-specific peptides. Top: CD8+ (CTL) immune response. Percentage of cytokine expressing T cells is shown. Bottom: CD4+ (TH1) immune response. Percentage of cytokine expressing T cells is shown. Each dot represents a single mouse, the bar is the geometric mean.

FIG. 8 CD8+ (CTL) immune response for IFNγ. Percentage of cytokine expressing T cells is shown. Each dot represents a single mouse, the bar is the geometric mean.

FIG. 9A and 9B Top: is a map of an anticancer amplicon expression vector vaccine encoding for dTERT. Bottom: Is a map of an amplicon anticancer amplicon expression vector vaccine encoding for a consensus sequence for telomerase reverse transcriptase (TERT).

FIG. 10 is a map of an anticancer amplicon expression vector vaccine encoding neoantigens associated with a MC38 colon carcinoma cell line. Each unlabeled separate box in the map is an ORF encoding an identified neoantigen associated with the MC38 colon carcinoma cell line.

FIG. 11 shows the antitumoral effect of an anticancer amplicon expression vector vaccine encoding a consensus sequence for TERT in a CT26 mouse tumor model applied in a prophylactic versus a therapeutic setting. The antitumoral effect of a plasmid encoding the same consensus sequence for TERT as the anticancer amplicon expression vector is also shown. The therapeutic setting was accompanied by immune checkpoint inhibitor (ICI) therapy consisting of PD-1 and CTLA-4. All vectors were administered via electroporation.

FIG. 12 is a scatter plot showing immunogenicity results of an anticancer amplicon expression vector vaccine encoding a consensus sequence for TERT versus a plasmid encoding the same consensus sequence for TERT and a control when administer in conjunction with ICI therapy consisting of PD-1 and CTLA-4. All vectors were administered via electroporation.

FIG. 13 shows the antitumoral effect in a MC38 mouse tumor model of an anticancer amplicon expression vector vaccine encoding neoantigens associated with a MC38 colon carcinoma cell line applied in a prophylactic and a therapeutic setting. The antitumoral effect of a plasmid encoding the same neoantigens as the anticancer amplicon expression vector is also shown. The therapeutic setting was accompanied by ICI therapy consisting of PD-1 and CTLA-4. All vectors were administered via electroporation.

FIG. 14 is a scatter plot showing immunogenicity results of an anticancer amplicon expression vector vaccine encoding neoantigens associated with a MC38 colon carcinoma cell line versus a plasmid encoding the same neoantigens and a control when administer in conjunction with ICI therapy consisting of PD-1 and CTLA-4. All vectors were administered via electroporation.

### DETAILED DESCRIPTION

Definitions

The term “amplicon” as used herein means a linear polynucleotide DNA or RNA molecule that is the product of an enzymatic or chemical-based amplification event or reaction. As used herein, amplicons are not derived from or produced via bacterial plasmid propagation and no not comprise plasmid derived DNA. An amplicon may be single or double stranded. Enzymatic or chemical-based amplification events or reactions include, without limitation, the polymerase chain reaction (PCR), loop mediated isothermal amplification, rolling circle amplification, nucleic acid sequence base amplification, and ligase chain reaction or recombinase polymerase amplification.

The term “continuous flow PCR device” means a PCR device as disclosed in U.S. Pat. No. 8,293,471, U.S. Pat. No. 8,986,982 and U.S. Pat. No. 8,163,489.

The term “large-scale PCR production” means a PCR reaction wherein the reaction vessel volume is greater than 0.0007 liters.

The term “episomal” means a DNA polynucleotide that is independent from a cell's chromosomal DNA. An episomal DNA polynucleotide may reside in a cell's nucleus.

The term “expression” means the transcription and/or translation of an expression cassette. The term “expression cassette” means a DNA sequence consisting of one or more genes and the sequences controlling their expression. At a minimum, an expression cassette shall include a promoter (or other expression control sequence), an open reading frame (ORF) and optionally, a terminator.

The term “amplicon expression vector” means an amplicon comprising one or more expression cassettes. As used herein, an amplicon expression vector must be the product of an enzymatic or chemical-based amplification event or reaction. An amplicon expression vector is not derived from or produced via bacterial plasmid propagation and is not comprised of plasmid derived DNA.

The term “next generation sequencing” (NGS) includes any form of high-throughput DNA or RNA sequencing. This includes, without limitation, sequencing by synthesis, sequencing by ligation, nanopore sequencing, single-molecule real-time sequencing and ion semiconductor sequencing.

The term “peptide modified” shall be construed to mean modification by peptides, polypeptides and/or proteins via complexing, bioconjugation, covalent bond, ionic bond, antibody-receptor binding or any combination of the foregoing.

The terminology used herein is for the purpose of describing particular embodiments only and is not intended to be limiting. The use of the alternative (e.g., “or”) should be understood to mean either one, both, or any combination thereof of the alternatives. As used herein, the indefinite articles “a” or “an” should be understood to refer to “one or more” of any recited or enumerated component.

Composition of Amplicon Expression Vectors

An amplicon expression vector may be comprised of an amplicon containing one or more expression cassettes. The amplicon expression vector can be single or double stranded, preferably double stranded. Optionally, an amplicon expression vector may also include one or more enhancers, a T7 or other secondary promoter, an open reading frame (ORF) for in-frame fusion tag (fusion ORF), an ORF for one or more CPP or NLS containing peptides, telomeric sequences, CPG open reading frames, chemical and/or peptide-based modifications and/or endonuclease protection moieties, including without limitation terminal phosphonothioate modifications.

The one or more ORFs of an amplicon expression vectors may be translatable into one or more target antigens, target neoantigens, other target polypeptides, target antigen receptors, target chimeric antigen receptors (CAR), or any other therapeutically relevant peptides, polypeptide, protein and/or RNA. Though derived from PCR or other enzymatic or chemical-based amplification event or reaction and not bacterial-plasmid propagation or plasmid derived DNA, the expression cassette and/or ORF sequences of an amplicon expression vector may be identical to the expression cassette and/or ORF sequences utilized in a plasmid-based expression vector.

The expression cassette and/or ORF sequences of an amplicon expression vector may also be modified for specific use in an amplicon expression vector. The ORF may be codon optimized and/or be comprised of a consensus sequence of one or more antigens or other immunogenic polypeptides.

Exemplary promoters include without limitation, hCMV, CMV, T7, EF1a, SV40 early promoter, SV40 later promoter, metallothionein promoter, murine mammary tumor virus promoter, Rous sarcoma virus promoter, polyhedron promoter, PGK1, Ubc, CAG, TRE, SSFV, UAS, Ac5 or another promoter shown effective for expression in eukaryotic cells. Exemplary enhancers include without limitation SV40 and CMV enhancer, as well as woodchuck hepatitis virus posttranscriptional regulatory element (WPRE), or other enhancer shown effective for expression in eukaryotic cells. Any terminator known in the art may be used. Exemplary terminators are SV40 polyadenylation/late polyadenylation signal, bovine growth hormone (bGH) polyadenylation signal and rabbit beta-globin (rbGlop) polyadenylation signal.

Modification of Amplicon Expression Vectors with Cell-Penetrating Peptides

One or more amplicon expression vectors may be modified via the incorporation of one or more cell-penetrating peptides (CPP). CPPs are capable of acting as a powerful transport vector for the intracellular delivery of amplicon expression vectors though the cell membrane, and in certain cases, into the cell nucleus. CPPs, without limitation, may be hydrophilic, polycationic, amphiphilic or contain a periodic sequence. His tagged WWRs, His tagged MGP or His tagged MGP/Rs may be used as CPPs.

CPP-mediated delivery of amplicon expression vectors necessitates proper conjunction of the CPPs with the payload amplicon. A preferred method of conjugation is the covalent bonding of CPPs to the amplicon expression vector. Covalent conjugation of one or more CPPs to an amplicon expression vector has the advantage of inherent proximity of the CPPs to the cargo amplicon. Covalent conjugation may occur during or after a PCR or other enzymatic/chemical based amplification reaction. Covalent conjugation may occur within a continuous flow PCR system, large-scale PCR production, or a non-continuous flow PCR system. Covalent conjugation may be accomplished via disulfide bonds, amine bonds, or specific linkers formulated to facilitate the release of the amplicon expression vector once internalized into a cell. The covalent conjugation of one or more CPPs to amplicon expression vectors may occur via the CPP's C- or N-terminal such that the CPPs' binding properties to a cells transport and other proteins are not affected

The amplicon expression vectors may be designed to facilitate covalent conjunction of CPPs at their 5′ and/or 3′ termini. In one embodiment, amplicon expression vectors with amine modified 5′ and/or 3′ termini may be manufactured via PCR to facilitate terminus covalent bonding of one or more CPPs to an amplicon. 5′ modification may be most compatible with introduction of CPPs during PCR-based manufacturing. Exemplary amine modifiers include DMS(O)MT-Amino-Modifier-C6, 5′ Amino Modifier C6, internal amino-c6-dT, 3′ Amino Modifier, Amino-Modifier-C3-TFA, Amino-Modifier-C12, Amino-Modifier-C6-TFA, Amino-dT, Amino-Modifier-5, Amino-Modifier-C2-dT, Amino-Modifier-C6-dT, 3′-Amino-Modifier-C7. Amine termini modification of amplicon expression vectors may be accomplished via PCR amplification with amine modified primers. Non-amine 3′ and/or 5′ termini modifiers may also be used to aid in terminus covalent bonding of CPPs. Exemplary non-amine modifiers include glycerol, phosphate, thiazolidine, oxime, hydrazine or thiol.

Covalent conjugation of CPPs to amine modified termini or other termini modified amplicon expression vectors may be accomplished in the reaction vessel of continuous flow PCR device, non-continuous flow PCR device or large-scale PCR production, or in separate vessel outside of a PCR device reaction vessel. In a continuous flow PCR device, covalent conjugation to amine modified termini or other termini modified amplicon expression vectors may be accomplished by the introduction of one or more CPPs into the PCR reaction at a predetermined time or location in the continuous flow pathway, such that covalent conjugation with the modified termini occurs in the same reaction vessel as the PCR amplification reaction. This will result in amplicon expression vectors with covalently conjugated CPPs at the conclusion of the continuous flow PCR device amplification cycle. The CPPs may be introduced during a PCR amplification reaction or after, but before the amplicons are deposited out of the reaction vessel. In a non-continuous flow PCR device, amine modified termini or other termini modified amplicon expression vectors may be covalently conjugated with one or more CPPs either in the PCR reaction's individual reaction vessels (e.g. each well of a 96 well plate or other discrete PCR reaction vessel of a large-scale PCR production) or the amplicons can be pooled and introduced to one or more CPPs in a separate vessel. The amine modified termini or other termini modified amplicon expression vector may be designed such that the covalent conjugation of one or more CPPs does not interfere with the biological activity of the amplicon expression vectors once transfected into a target cell. Amplicon expression vectors with one or more covalently conjugated CPPs may be used in conjunction with all known electroporation techniques, including all known in vivo and ex vivo electroporation techniques, including without limitation, minimally-invasive electroporation devices configured for intradermal and/or intramuscular electroporation.

Peptide nucleic acid (PNA)-mediated PCR clamping may also be used to hybridize a PNA linked to a CPP to an amplicon expression vector in lieu of, or in addition to, covenant conjugation.

The CPPs may also be complexed with amplicon expression vectors via electrostatic/ionic, hydrophobic or other non-covalent interactions via bulk mixing of one or more CPPs and amplicon expression vectors. This mixing may be accomplished in the reaction vessel of a continuous flow PCR device, a large-scale production or non-continuous flow PCR device during or after a PCR amplification reaction to form amplicon/CPP complexes. The CPPs may also be bulk mixed with the amplicon expression vectors continuously throughout a continuous flow PCR amplification cycle. Bulk mixing is possible in a non-continuous flow PCR device or during large-scale PCR production within each discrete reaction vessel. Complexing may also be accomplished after aggregation of amplicons in a separate vessel. The sequence of the amplicon expression vectors may be designed to facilitate increased or advantageous complexing of CPPs via electrostatic, hydrophobic or other non-covalent binding conditions. CPP's may also be bound to G-quadruplex structures added to amplicon expression vectors via modified primers via conjugation with an antibody that binds to the G-quadruplex structure.

ORFs for desired one or more CPPs may also be placed in-frame within the amplicon expression vector, and when transcribed and translated, will produce one or more CPPs that will associate with the amplicon expression vector, thereby increasing transfection efficiency. The in-frame CPP ORFs may be driven by a separate promoter than the amplicon expression vector's therapeutic OFR (i.e. the open reading frame for the desired antigen or other therapeutic peptide or protein) such that separate transcription and translation events are possible. The in-frame DNA ORFs for one or more CPPs may be driven by a separate T7 promoter such that in vitro transcription and translation of the one or more CPP ORFs can occur before transfection of the amplicon expression vector into a subject's cells. The CPPs expressed via in vitro transcription and translation will associate with the amplicon expression vector and increase transfection efficiency. CPPs may also be linked to one more nanoparticles and then covalently bound to or complexed with amplicon expression vectors.

Modification of Amplicon Expression Vectors with Nuclear Localization Sequence Containing Peptides

Amplicon expression vectors may be modified via the incorporation of peptides containing on or more nuclear localization sequences (NLSs) to provide for efficient transport to the cell nucleus for translation, and to minimize time spent in the cytosol of target cells post transfection. The use of NLS containing peptides is advantageous in both dividing and non-dividing target cells. The NLSs may be monopartite or bipartite, or take the form of other non-classical NLSs. The NLS containing peptides may be complexed with amplicon expression vectors via electrostatic interactions during or after PCR amplification in the same fashion as CPPs as disclosed herein. In addition, NLS containing peptides may also be conjugated to amplicon expression vectors via random covalent attachment and/or by site specific covalent conjugation. Site specific covalent conjugation may be accomplished via the use of amplicon expression vectors with amine modified termini or other modified termini, or through the use of PNA mediated PCR clamping (hybridization) of a PNA linked to a NLS containing peptide in the same fashion as CPPs, as disclosed herein. An exemplary form of site specific covalent conjugation is attachments of one or more NLS containing peptides at the 5′ and/or 3′ termini of the amplicon expression vector. The addition of one or more NLS containing peptides to amplicon expression vectors may be undertaken during or after PCR amplification in the same fashion as CPPs. The covalent conjugation of a NLS containing peptide to amplicon expression vectors may occur via the NLS containing peptide's C- or N-terminal, such that the NLS containing peptide binding properties to a cell's transport proteins is not affected. NLS containing peptides may also be linked to one more nanoparticles and then covalently bound to or complexed with amplicon expression vectors

Nuclear transport of the amplicon expression vector may also be facilitated via the addition of nuclear targeting sequences (DTSs). A DTS can be placed in-frame within an amplicon expression vector's ORF, and when transcribed and translated, will produce one or more NLS containing peptides that will associate with the amplicon expression vector, thereby increasing nuclear transport efficiency. An exemplary DTS is the SV40 enhancer.

ORFs for one or more desired NLS containing peptides (including but not limited to nuclear targeting sequences) may also be placed in-frame within the amplicon expression vector, and when transcribed and translated, will produce one or more NLS containing peptides that will associate with the amplicon expression vector, thereby increasing nuclear transport efficiency. The one or more in-frame NLS containing peptide ORFs may be driven by a separate promoter than the therapeutic open reading frame within the amplicon expression vector (i.e. the open reading frame for the desired antigen or other therapeutic peptide or protein) such that separate transcriptions and translation events are possible. One or more NLS containing peptide ORFs in-frame of an amplicon expression vector may be driven by a separate T7 promoter such that in vitro transcription and translation of the one or more NLS containing peptide ORFs can occur before transfection into a subject's cells. The one or more NLS containing peptides expressed via in vitro transcription and translation will then associate with the amplicon expression vector and increase nuclear transport efficiency.

Amplicon Expression Vectors Complexing with Cationic Polymers, Branched Dendrimers, Cationic Liposomes

One or more amplicon expression vectors may be complexed with various transfection agents to increase transfection efficiency. Complexing may be desirable with or without electroporation. Polycation/DNA complexes (polyplexes), which are formed between cationic polymers and DNA though electrostatic interactions, can be created with the amplicon expression vectorsvia the controlled introduction of one or more cationic polymers. Said introduction may occur during or after PCR amplification. In certain embodiments, the introduction of one or more cationic polymers may occur within the PCR reaction of a continuous flow PCR device. Alternatively, the introduction of one or more cationic polymers may occur in the discrete reaction vessels of a non-continuous flow PCR device or large-scale PCR production, or within a separate vessel after amplicon expression vector assemblage.

Exemplary cationic polymers include polyamines, poly(2-dimethylaminoethyl methacrylate) (pDMAEMA), poly-L-lysine (pLL), diethylaminoethyldextran (DEAE-dextran), Polyethyleneimine (PEI), Poly(2-dimethylamino)ethyl methacrylate (PDMAEMA). In addition, biodegradable cationic polymers maybe be utilized to form polyplexes. These polyplexes formed from biodegradable cationic polymers are less likely to lead to intercellular accumulation of the polymer, leading to less toxicity, and allows for the intercellular degradation of the polymer, thereby releasing the complexed amplicon expression vector into the cytosol and/or nucleus. Exemplary biodegradable cationic polymers include synthesized polyphosphazenes, Poly(4-hydroxy-1-proline ester), Poly(γ-(4-aminobutyl)-1-glycolic acid), poly(2-dimethylaminoethylamine)phosphazene and poly(2-dimethylaminoethanol) and phosphazene.

Likewise, lipoplexes (cationic liposome-DNA complexes) can also be formed with amplicon expression vectors. Exemplary cationic liposomes are comprised of 3β-[N-(N′,N′-dimethylaminoethane) carbamoyl] cholesterol (DC-Chol) and 1,2-dioleoyl-sn-glycero-3-phosphoethanolamine (DOPE), and O,O′-ditetradecanoyl-N-(α-trimethyl ammonioacetyl) diethanolamine chloride and cholesterol.

Synthetic branched dendrimers, which are comprised of three dimensional, highly branched, mono-disperse macromolecules of nanometer dimensions (such as Starburst Polyamidoamine (PAMAM)) may also be complexed with the amplicon expression vectors. Like cationic polymers, complexing of cationic liposome and/or synthetic branched dendrimers with amplicon expression vectors may occur during or after PCR amplification. The complexing may occur within the PCR reaction of a continuous flow PCR device, within in the discrete reaction vessels of a non-continious flow PCR device or large-scale PCR production, or within a separate vessel after amplicon expression vector assemblage.

Modification of Amplicon Expression Vectors with In-Frame Fusion Tags

The amplicon expression vector may include an in-frame fusion ORF that is translatable into one or more fusion tags such as small ubiquitin-related modifier (SUMO), said SUMO optimized for human and/or eukaryotic expression, ubiquitin (Ub), maltose binding protein (MBP), glutathione S-transferase (GST), thioredoxin (TRX), Strep-tag, Strep-tag II and NUS A. A fusion tag is a short peptide, protein domain, or entire protein that can be fused to a target protein. When the ORF and the fusion ORF of an amplicon expression vector are both translated, the target antigen or other protein with a fusion tag is produced creating a fusion protein.

Fusion tags have been used widely in heterologous expression systems as a technique to stabilize the recombinant product against proteolysis, increase the translational initiation efficiency or to serve as an affinity handle for the purification of the protein. A fusion tag is generally a protein or a peptide located either on the C- or N-terminal end of the target protein, which facilitates features such as improved solubility, detection, purification, localization or expression. They can improve the variable yield and poor solubility of many recombinant proteins. Proper design and judicious use of the right fusion tag can enhance the solubility and promote proper folding of the protein of interest, leading to recovery and/or expression of more functional antigen or other protein. It may be desirable to remove the tag from the target antigen or other protein after expression by means of cleaving tags.

Through the use of one or more fusion tags, the expression level of an antigen, neoantigen or other protein encoded by the amplicon expression vector may be increased or modified as necessitated by the specific antigen-specific immune response desired in the subject. In one embodiment, an amplicon expression vector containing an ORF for canine telomerase reverse transcriptase (“dTERT”), or a consensus sequence for telomerase reverse transcriptase (“TERT”), or other tumor associated antigens, including neoantigens, and a fusion ORF for a fusion tag may be utilized to provide high level expression of the encoded antigen(s) to increase the efficacy of the antigen-specific immune response. The fusion tags may be removed from the target antigen or protein after expression by means of appropriate cleaving tags.

Exonuclease Protection

Amplicon expression vectors may also include protective features to reduce degradation by exonuclease or other factors. In some embodiments, a protective feature is introduced at the 5′ and/or 3′ termini of the amplicon expression vectors. In one embodiment, a length of noncoding DNA sequence extending beyond the promoter and/or terminator is added. This noncoding DNA may be G-quadruplex structures or may be other noncoding DNA of a known sequence and length. G-quadruplex structures may be added to amplicon expression vectors via the use of modified PCR primers. In another embodiment, phosphorothioate-modification may be used to protect amplicon expression vectors against exonuclease and/or other degradation. Said phosphorothioate-modification may be accomplished via phosphorothioate modified PCR primers used during PCR-based manufacture of the amplicon expression vectors, which may provide for the protection of the 5′ and/or 3′ termini of an amplicon expression vector against degradation by exonuclease or other intracellular factors. Peptide nucleic acid (PNA) sequences may also be used to protect the 3′ and/or 5′ termini of amplicon expression vectors.

Protective termini modification of amplicon expression vectors may also be used to provide for “tunable” episomal persistence of an amplicon expression vector, such that the duration of expression by a subject's cell can be tailored for specific therapeutic applications. Terminal G-quadruplex structures and/or phosphorothioate-modification may be used. The addition of terminal (or extending beyond the promoter and/or terminator) noncoding DNA sequences may also be used.

Assembly of Peptide-Modified Amplicon Expression Vectors

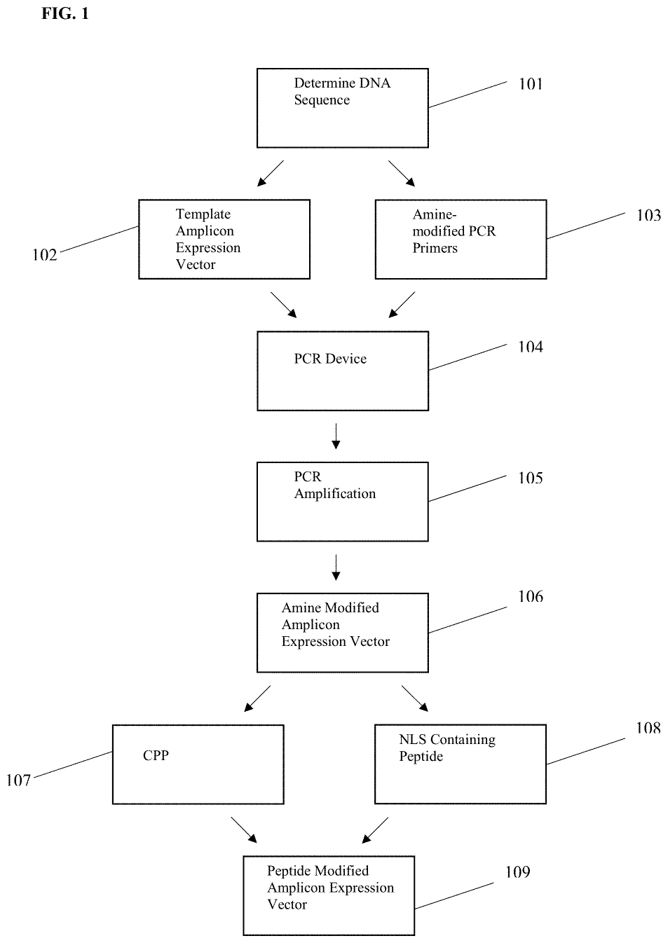

Turning to FIG. 1, a flow diagram showing the assembly of termini peptide-modified amplicon expression vectors via a PCR device is shown. First, the DNA sequence of the amplicon expression vector is determined (101) which will include a promoter, on or more ORFs, and terminator. It may optionally include a fusion ORF or other secondary ORF (such as for CPPs or NLS containing peptides) and/or one or more enhancers. Once the sequence is known, a template amplicon expression vector (102) is created via DNA synthesis. Appropriate amine-modified PCR primers (103) for the specific sequence of the template amplicon expression vector (102) are generated via oligonucleotide synthesis. The template amplicon expression vector (102) and the amine-modified primers (103) are introduced to a PCR device (104) whereby the template amplicon expression vector is exponentially amplified via PCR (105) to create amplicon expression vector with amine modified 3′ and/or 5′ termini (106). After PCR amplification, the amplicon expression vectors are introduced to CPPs (107) which may be formulated to covalently conjugate to the amine modified 3′ and/or 5′ termini of the amplicon expression vectors. Introduction to the CPPs (107) may occur within or outside of the PCR device reaction vessel. Contemporaneously or after introduction of the CPPs, NLS containing peptides (108) may be introduced to the amplicon expression vectors. The NLS containing peptides (108) may be formulated for non-covalent complexing or for covalent conjugation with the amine modified amplicon expression vectors (106). Covalent conjugation of the NLS containing peptides (108) may occur at the amine modified 3′ and/or 5′ termini of the amplicon expression vectors. The 3′ and 5′ termini of the amplicon expression vectors may be modified with different amines or different chemical compounds such that one termini may bind one or more CPPs while the other termini may bind one or more NLS containing peptides.

Upon completion of the PCR reaction and subsequent peptide modification, the now peptide-modified amplicon expression vectors (109) are purified and ready for use in the transfection of a target cell for expression of the desired peptide, antigen, polypeptide or protein.

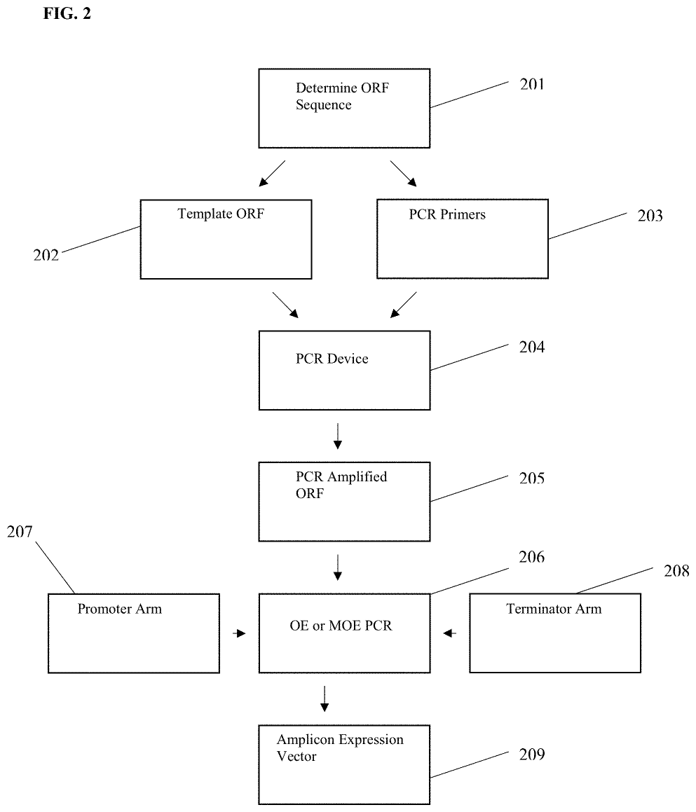

In FIG. 2, an alternative embodiment of amplicon expression vector assembly is shown. The DNA sequence of one or more desired ORFs (201) is determined, and a template ORF (202) is created via DNA synthesis. The ORF template (202) is PCR amplified with appropriate non-chemically modified primers (203) via PCR (204). The PCR amplified ORF (205) is then subject to an overlap extension (OE) or multiple overlap extension (MOE) PCR reaction (206) wherein pre-manufactured promoter arms (207) and pre-manufactured terminator arms (208) are added to the PCR amplified ORF (205) via hybridization to create amplicon expression vectors (209). The promoter arm (207) may contain a promoter sequence, and optionally, a fusion ORF, a secondary ORF or secondary expression cassette, G-quadruplex structures, enhancer, and/or one or more covalently conjugated CPPs and/or NLS containing peptides. The terminator arm (208) may contain a termination sequence, and optionally, a fusion ORF, a secondary ORF or secondary expression cassette, G-quadruplex structures, enhancer, a sequence translatable into a poly-a tail or other structural element and/or one or more covalently conjugate CPPs and/or NLS containing peptides. The promoter arm and/or terminator arm may also be phosphorylated. The promoter and/or terminator arms may also contain one or more ORFs or expression cassette for one or more CPPs or NLS containing peptides driven by a T7 or other secondary promoter.

Various polymerases, with different error rates, may be used for the PCR-based production of the amplicon expression vectors. Error rate may be therapeutic application dependent or subject dependent. Error rates of a polymerases may be lower or higher than the error rate of human or another organism's DNA replication. The polymerase may be a proof reading or non-proof reading polymerase. Exemplary polymerases include Taq polymerase (2×10⁻⁴ error/bp), Pfu polymerase (1.3×10⁻⁶), Q5® polymerase (New England BioLabs) (5×10⁻⁷ error/bp), Phusion® polymerase (New England BioLabs) (4×10⁻⁶ error/bp) BIOLASE™ polymerase (Bioline) (7×10⁻⁵ approximate error/bp), MyFi™ polymerase (Bioline) (7×10⁻⁵ approximate error/bp) RANGER™ polymerase (Bioline) (1×10⁻⁶ approximate error/bp), Velocity polymerase (Bioline) (4.4×10⁻⁷ error/bp).

Amplicon Expression Vectors as DNA Vaccines

Amplicon expression vectors may be used as a DNA vaccine to elicit a wide range of antigen-specific immune responses. When utilized as a DNA vaccine, the amplicon expression vectors encode a desired antigen or other immunogenic polypeptide for in vivo expression within a subject's cells. The antigen may be a peptide or protein that causes an immune response. The antigen may trigger production of antibodies by the subject's immune system for therapeutic effect. The amplicon expression vector may induce innate immunity activation and/or adaptive immune responses.

The antigen or other immunogenic polypeptide expressed by an amplicon expression vector may be any molecule or molecule fragment that can be bound by a major histocompatibility complex (MHC) and presented to a T-cell receptor. The antigen may also be an immunomodulatory molecule or immunogen (collectively “immunogenic peptides”). The antigen may be associated with a virus, bacterial infection, fungal infection, parasitic infection, influenza, autoimmune disease, cancer, allergy, asthma, or an autoimmune disease. The antigen may affect a mammal, which may be a human, chimpanzee, dog, cat, horse, cow, mouse, or rat. The antigen may be contained in a protein from a mammal, which may be a human, chimpanzee, dog, cat, horse, cow, pig, sheep, mouse, or rat.

In addition, antigens and other immunogenic polypeptides expressed by the amplicon expression vectors may be used to prevent or treat, i.e., cure, ameliorate, or lessen the severity of cancer including, but not limited to, cancers of oral cavity and pharynx (i.e., tongue, mouth, pharynx), digestive system (i.e., esophagus, stomach, small intestine, colon, rectum, anus, anal canal, anorectum, liver, gallbladder, pancreas), respiratory system (i.e., larynx, lung), bones, joints, soft tissues (including heart), skin, melanoma, breast, reproductive organs (i.e., cervix, endometrium, ovary, vulva, vagina, prostate, testis, penis), urinary system (i.e., urinary bladder, kidney, ureter, and other urinary organs), eye, brain, endocrine system (i.e., thyroid and other endocrine), lymphoma (i.e., hodgkin's disease, non-hodgkin's lymphoma), multiple myeloma, leukemia (i.e., acute lymphocytic leukemia, chronic lymphocytic leukemia, acute myeloid leukemia, chronic myeloid leukemia).

Examples of viral antigens and other viral immunogenic polypeptides include, but are not limited to, denovirus polypeptides, alphavirus polypeptides, calicivirus polypeptides, e.g., a calicivirus capsid antigen, coronavirus polypeptides, distemper virus polypeptides, Ebola virus polypeptides, enterovirus polypeptides, flavivirus polypeptides, hepatitis virus polypeptides, herpesvirus polypeptides, poliovirus polypeptides and rabies virus polypeptides.

Examples of bacterial antigens and other bacterial immunogenic polypeptides include, but are not limited to, Salmonella polypeptides, Listeria polypeptides, Actinomyces polypeptides, Bacillus polypeptides, Bacteroides polypeptides , Bartonella polypeptides, Borrelia polypeptides, Brucella polypeptides, Campylobacter polypeptides, Capnocytophaga polypeptides, Chlamydia polypeptides, Clostridium polypeptides, Corynebacterium polypeptides, Coxiella polypeptides, Bordetella polypeptides, Dermatophilus polypeptides, Enterococcus polypeptides, Ehrilichia polypeptides, Escherichia polypeptides, Francisella polypeptides, Peptococcus polypeptides, Fusobacterium polypeptides, Haemophilus polypeptides, Leptospira polypeptides, Helicobacter polypeptides, Klebsiella polypeptides, L-form bacteria polypeptides, Mycobacteria polypeptides, Proteus polypeptides, Mycoplasma polypeptides, Neisseria polypeptides, Neorickettsia polypeptides, Nocardia polypeptides, Pasteurella polypeptides, Peptostreptococcus polypeptides, Pneumococcus polypeptides, Pseudomonas polypeptides, Rickettsia polypeptides, Rochalimaea polypeptides, Haemobartonella polypeptides, Shigella polypeptides, Staphylococcus polypeptides, Streptococcus polypeptides, Treponema polypeptides, and Yersinia polypeptides. Examples of fungal antigens and other fungal immunogenic polypeptides include, but are not limited to, Leishmania polypeptides, Microsporidia polypeptides, Neospora polypeptides, Nosema polypeptides, Pentatrichomonas polypeptides, Plasmodium polypeptides, Pneumocystis polypeptides, Sarcocystis polypeptides, Schistosoma polypeptides, Theileria polypeptides, Toxoplasma polypeptides, and Trypanosoma polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Rhinosporidium polypeptides, Rhizopus polypeptides, Scolecobasidium polypeptides, Sporothrix polypeptides, Stemphylium polypeptides, Trichophyton polypeptides and Trichosporon polypeptides.

Amplicon expression vectors may be delivered to a subject via an electroporation device or a minimally-invasive electroporation device configured for intradermal and/or intramuscular electroporation. Exemplary electroporation devices, and methods of their use are described in U.S. Pat. No. 6,520,950; U.S. Pat. No. 7,171,264; U.S. Pat. No. 6,208,893; U.S. Pat. No. 6,009,347; U.S. Pat. No. 6,120,493; U.S. Pat. No. 7,245,963; U.S. Pat. No. 7,328,064; and U.S. Pat. No. 6,763,264, the contents of each of which are herein incorporated by reference. The Vet-ePorator Device EPS01 (Evvivax, Italy) or similar device may also be utilized. The Amplicon expression vector may also be administered to a subject via transdermal patches, needle arrays, needle injection, needle-free injection and topical application, or any combination thereof. Administration may also be in combination with one or more adjuvants known in the field of immunization.

Amplicon Expression Vectors as DNA Cancer Vaccines

Amplicon expression vector may be utilized as DNA cancer vaccines. Targets may include one or more tumor-specific antigens (TSA), tumor associated antigens (TAA), cancer testis antigens (CTAs), tumor neoantigens, mutational associated neoantigens and/or oncoviral, oncofetal, lineage-restricted, and/or over-expressed tumor antigens. Specific TAA include, without limitation, hTERT, TERT, a consensus sequence of TERT, dTERT, HER-2, BIRCS, MAGE, NY-ESO-1, Melan-A/MART-1, GP100, Tyrosinase, TRP1/2, PSA, PAP, PSMA, Mamoglobin A, CEA, HPV E6/E7.

In an embodiment, a universal cancer DNA vaccine may be produced comprising an amplicon expression vector encoding a consensus sequence of telomerase reverse transcriptase (TERT). High levels of human telomerase reverse transcriptase (hTERT) are detected in over 85% of human cancers. Canine telomerase reverse transcriptase (dTERT) is similarly characterized. hTERT/dTERT expression correlates with telomerase activity, which may be a requirement for tumor survival. hTERT/dTERT expression is associated with little loss of telomere length and accounts for the unlimited proliferative capacity of cancer cells. Loss of hTERT/dTERT activity will lead to hTERT/dTERT positive tumor cell death by apoptosis. As such, hTERT and dTERT are attractive TAA for a universal cancer vaccine in human and canines, respectively.

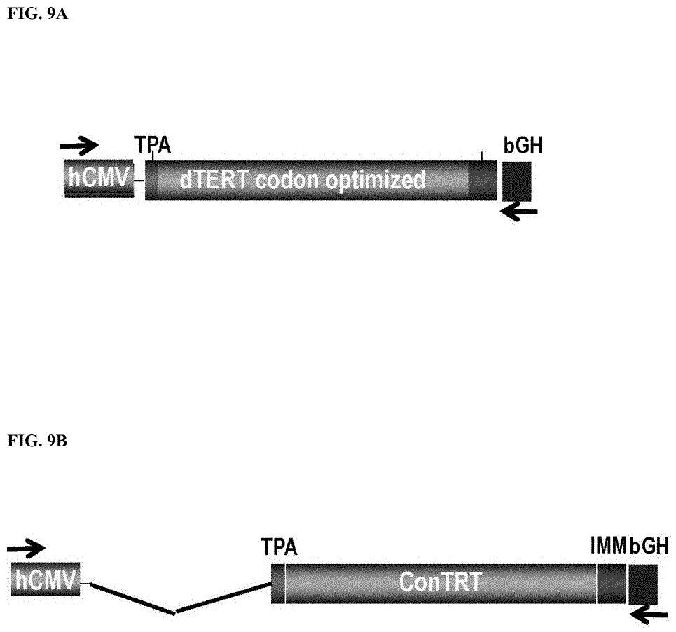

In an embodiment, and as shown in FIG. 9A, a universal canine anticancer vaccine may be comprised of an amplicon expression vector, said amplicon expression vector containing: (a) hCMV promoter; (b) optionally, an ORF for one or more tissue plasminogen activator (TPA) signal peptides; (c) an ORF for dTERT codon optimized; (d) optionally, an immunomodulator; and (e) and an appropriate terminator such as bGH. The immunomodulator may be any known immunomodulator known in the art, including without limitation, those isolated from a protozoan. The TPA may be human TPA or other form of TPA.

In another aspect of the invention, and as shown in FIG. 9B, a universal anticancer vaccine may be comprised of an amplicon expression vector, said amplicon expression vector containing: (a) hCMV promoter; (b) optionally, an ORF for one or more tissue plasminogen activator (TPA) signal peptides; (c) an ORF for a consensus sequence of TERT; (d) optionally, an immunomodulator; and (e) and appropriate terminator such as bGH. The immunomodulator may be any known immunomodulator known in the art, including without limitation, those isolated from a protozoan.

The anticancer vaccines disclosed herein, when produced as an amplicon expression vector do not utilized plasmid DNA or plasmid-derived DNA, and thus, are free of any endotoxin contamination and/or any bacterially derived DNA, including without limitation, bacterially derived DNA conferring antibiotic resistance.

In one aspect of the invention, a method for prophylactically and/or therapeutically preventing, ameliorating or treating cancer in a subject via a amplicon expression vector vaccine is disclosed, said method comprising: (a) administering to a subject in need thereof an amplicon expression vector comprising: (i) a promoter; (ii) optionally, an ORF for one or more tissue plasminogen activator (TPA) signal peptides; (iii) an ORF for a consensus sequence of TERT; (iv) optionally, an immunomodulator; and (v) an appropriate terminator such as bGH; and (b) said amplicon expression vector administered to the subject via electroporation. The promoter may be any appropriate promoter known in the art and may be subject specific. The promotor may be hCMV.

In yet another aspect of the invention, a method for prophylactically and/or therapeutically preventing, ameliorating or treating cancer in a canine subject via a amplicon expression vector vaccine is disclosed, said method comprising: (a) administering to a subject in need thereof an amplicon expression vector comprising: (i) a promoter; (ii) optionally, an ORF for one or more tissue plasminogen activator (TPA) signal peptides; (iii) an ORF for codon optimized dTERT; (iv) optionally, an immunomodulator; and (v) and appropriate terminator such as bGH; and (b) said amplicon expression vector administered to the subject via electroporation. The promoter may be any appropriate promoter known in the art and may be subject specific. The promotor may be hCMV.

Amplicon Expression Vectors as Neoantigen-Target Cancer Vaccine

In one aspect of the invention, a DNA vaccine targeting one or more tumor neoantigens or other epitopes may be produced via amplicon expression vectors. Neoantigens arise from somatic mutations that differ from wild-type antigens and are specific to each individual subject, or a sub-population of subjects, which provide tumor specific antigen targets. Neoantigens arise from tumor mutation-based peptide sequences and are found only in tumor cells, and thus are not subject to self-tolerance, have a decreased risk of generated autoimmunity and on-target off-tumor effects.

One or more tumor neoantigens may be identified in a subject by differential sequencing of a subject's tumor versus wild-type samples, using exome/genome sequences and RNAseq analysis, and the assistance of artificial intelligence, machine learning, predictive algorithms or the like. Through this method, a DNA cancer vaccine comprising one or more amplicon expression vectors encoding the subject specific identified neoantigens can be designed and produced via a plasmid-derived DNA free workflow that will elicit an antigen-specific immune response to said neoantigens, resulting in a cancer vaccine with limited on-target off-tumor effects and high efficacy without the risk of endotoxin or bacterial DNA contamination. A single amplicon expression vector may encode one neoantigen or it may encode for more than one neoantigen. In one embodiment, one or more ORFs of an amplicon expression vector may encoding human tissue plasminogen activator (TPA) signal peptides, a helper epitope from Tetanus toxin (p30), ubiquitin, small ubiquitin-like modifier (SUMO), furin or other cleavage sites, membrane-translocating sequences (MTS) from HIV-1 and E. Coli enterotoxin B subunit, an immunomodulator, as well as one or more neoantigens or other epitopes. A fusion protein may or may not be produced. The amplicon expression vector may be comprised of a single polynucleotide molecule or several polynucleotide molecules linked together with cleavable or non-cleavable linkers.

Such “personalized” neoantigen DNA cancer vaccines are well suited for amplicon expression vectors and their method of rapid manufacture. In an embodiment, a subject's tumor neoantigens are determined via differential sequencing, RNAseq analysis, bioinformatics software or the like. The DNA sequence for the desired one or more neoantigens or other identified epitopes is determined and an ORF sequence encoding for the one or more neoantigens is created via plasmid-DNA free synthetic DNA synthesis. The ORF sequence is may be coupled with preexisting promoter and terminator arms to create an amplicon expression vector as described in FIG. 2. The neoantigen or other epitope ORF may also be combined with an appropriate promoter and termination, as well as other elements disclosed herein to accomplish therapeutically effective expression of the neoantigens or other epitope. This neoantigen or other epitope encoding amplicon expression vector is then amplified via PCR to create a therapeutic dose. With modern next generation sequencing (e.g. Illumina NovaSeq 6000), efficient bioinformatics platforms, and rapid high-fidelity synthetic DNA synthesis, is it possible to create a therapeutic dose of personalized neoantigen encoding amplicon expression vectors in under 48 hours. In another embodiment, DNA cancer vaccine amplicon expression vectors may be based upon neoantigens or other epitopes known to be associated with a specific type of cancer or may be mutanome and/or neoepitope directed.

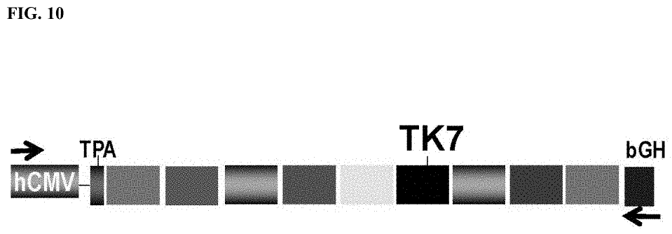

In one aspect, an amplicon expression vector comprising one or more ORFs encoding one or more neoantigens, said amplicon delivered to a subject via electroporation, is used as a cancer vaccine. The desired neoantigens to be expressed by the ORFs included in the amplicon expression vector may be obtained via differential sequencing and bioinformatics software, RNAseq, HLA binding predictions, and/or epitopes known to be associated with a specific type of cancer. The ORFs encoding the neoantigens may be codon optimized and may encode for between 5 to 100 amino acids. Exemplary embodiments encode for between 7 and 27 amino acids per ORF. As shown in FIG. 10, expression of the ORFs contained in amplicon expression vector maybe driven by hCMV or other appropriate promoter. Exemplary neoantigens for a MC38 colon carcinoma cell line that may be encoded by ORFs contained in an amplicon expression vector are detailed in the chart below. Each neoantigen identified is represented by an ORF (each square) encoding for the neoantigen as shown on FIG. 10.

| | | WT Peptide | | | Neoantigen | | | | Imm. |
|---|---|---|---|---|---|---|---|---|---|
| # | Symbol | Seq | H2-Db | H2- | Seq | H2-Db | H2-Kb | DAI | Resp. |
| 11 | Tmem135 | FALMNRKAL | 12 | 1426 | FALMNLKAL | 12 | 681 | <5 | + |
| 12 | Aatf | MAPIDHTAM | 297 | 5239 | MAPIDHTTM | 90 | 6675 | >5 | − |
| 13 | Spire1 | SAIRSYQDV | 814 | 271 | SAIRSYQYV | 28 | 43 | >5 | + |
| 14 | Zbtb40 | KSFHFYCRL | 7827 | 4 | KSFHFYCPL | 1318 | 4 | <5 | − |
| 15 | Slc12a4 | LSAARYALL | 26,865 | 16 | LSASRYALL | 27,697 | 14 | <5 | − |
| 16 | Nfe2I2 | ASYSQVAHI | 4627 | 66 | ASYSLVAHI | 1309 | 23 | <5 | − |
| 17 | Herc6 | CGYEHTAVL | 7636 | 140 | CVYEHTAVL | 9017 | 91 | <5 | − |
| 18 | Copb2 | MSYFLQGKL | 6858 | 72 | MSYFLQGTL | 2039 | 51 | <5 | − |
| 19 | Reps1 | AQLPNDVVL | 89 | 9261 | AQLANDVVL | 16 | 8591 | >5 | + |
| 20 | Adpgk | ASMTNRELM | 6 | 2267 | ASMTNMELM | 4 | 1288 | <5 | + |

The amplicon expression vector may encode for 2 to 20, 2 to 10, 2 to 5, 4 to 8, 5 to 15 or more than 20 neoantigens. In certain aspects, the one or more ORFs are linked to other ORFs in an amplicon expression vector or other aspects of the amplicon expression vector construct by furin sensitive linkers or other cleavable linkers known in the art. The amplicon expression vector may alternatively not use furin sensitive or other cleavable linkers, and instead the one or more ORFs may subsist in a single polynucleotide molecule, said ORFs driven by one or more appropriate promoters. Said expression amplicon expression vector may also include ORFs for tissue plasminogen activator (TPA) signal peptides, a helper epitope from Tetanus toxin (p30), ubiquitin, small ubiquitin-like modifier (SUMO), furin or other cleavage sites, membrane-translocating sequences (MTS) from HIV-1 and/or E. Coli enterotoxin B subunit.

In one aspect, an amplicon expression vector encoding more than one neoantigen may be comprised of a single polynucleotide molecule without cleavable linkers, said amplicon comprised of the following elements : (a) one or more ORFs encoding for one or more desired cancer specific neoantigens; (b) a promoter; (c) optionally, a tissue plasminogen signal peptide; (d) optionally, an E. coli heat labile enterotoxin B subunit; (e) optionally, SUMO (f) optionally, an immunomodulator; and (g) a terminator. The promotor may be hCMV and the termination may be bGH.

In another aspect, a method for treating cancer, without the use of bacterial plasmid derived DNA, via the use of amplicon expression vectors comprised of one or more ORFs encoding cancer specific neoantigens is disclosed, said method comprising: (a) determining the target neoantigens; (b) assembling an amplicon expression vector template comprising one or more ORFs encoding the target neoantigens via plasmid DNA free gene synthesis assembly; (c) amplifying the amplicon expression vector template comprising ORFs encoding the target neoantigens via PCR; (d) purifying and concentrating the PCR produced amplicon expression vectors to quantifiable metrics necessary for an efficacious therapeutic or prophylactic dosage for a subject; and (e) administering the therapeutic or prophylactic dose of amplicon expression vectors comprising ORFs encoding the target neoantigens via electroporation.

The target neoantigens may be determined via any means known in the art, including, without limitation, differential sequencing of a subject's tumor versus wild-type samples, using exome/genome sequences, RNAseq analysis, and the assistance of artificial intelligence, machine learning, predictive algorithms or the like. In one aspect, the target neoantigens are determined via: (m) taking a tumor and blood sample from the subject; (n) performing exome sequencing, RNAseq and HLA typing on the subject's tumor and blood samples; (o) computer aided identification of the cancer specific and/or cancer associated MHCI/II epitopes via computer assisted methodologies; and (p) designing an ORF to encode the neoantigen correlating to the identified MHCI/II epitopes. Assembly of the amplicon expression vector template comprising the one or more ORFs encoding for the target neoantigens may be accomplished by any means known in the art, including, without limitation, any means of creating a DNA sequence via artificial gene synthesis and without the use of plasmid-derived DNA, which may include, photolithographic means, oligonucleotide synthesis, solid-phase DNA synthesis or any other means of plasmid-derived DNA-free gene synthesis. In an embodiment, the source of nucleotides is amidites, phosphoramidites and/or nucleoside phosphoramidites. In addition to the ORFs for the identified neoantigens, the amplicon expression vector may also include an appropriate promotor, such as hCMV and optionally, other elements disclosed herein, such as, without limitation, SUMO, TPA and linkers. Purification and concentration of the amplicon expression vectors may be accomplished via techniques known in the art, including, without limitation, high performance liquid chromatography (HPLC), filtration, ethanol precipitation and other analytical chemistry techniques. Electroporation may be accomplished via commercially available electroporation devices and methods, including, without limitation, those disclosed herein.

In one aspect, target neoantigens may be identified from circulating tumor cells or invasive circulating tumor cells (collectively “CTCs”). CTC's may be isolated from a patient and identified as disclosed in U.S. Pat. Nos. 7,785,810, 7,687,241 and 8,889,361, which are hereby incorporated by reference in their entirety. Once the CTCs are isolated and identified, target neoantigens can be determined therefrom using the neoantigen identification techniques described herein. Once identified, an amplicon expression vector-based vaccine containing one or more ORFs encoding the neoantigens can be produced and administered to a subject according to the methods of the instant invention.

Amplicon Expression Vectors as Anti-Cancer DNA Vaccines in Conjunction with ICIs

The anti-cancer amplicon expression vector vaccines disclosed herein may also be used in conjunction with immune check-point inhibitors (ICI) such as, without limitation, anti-PD-1 (PD-1), anti-PD-L1 (PD-L1) and anti-CTLA-4 (CTLA-4) antibodies. These antibodies may be administered to a subject as a separate monoclonal antibody co-therapy, or as one or more amplicon expression vectors encoded to express one or more ICIs in vivo.

In an embodiment, an amplicon expression vector may contain ORFs for one or more neoantigens and one or more ICIs. In an alternative embodiment, an amplicon expression vector may contain ORFs for hTERT, dTERT or a consensus sequence of TERT, as well as one or more ICIs. In another aspect, an amplicon expression vector may contain one or more ORFs encoding for neoantigens and one or more ORFs for one or more ICIs. An amplicon expression vector may contain one or more ORFs encoding for neoantigens may also be administered in conjunction with ICI co-therapies.

Having now generally described the invention, the same will be more readily understood through reference to the following examples, which are provided by way of illustration, and are not intended to be limiting of the disclosed invention, unless specified.

### EXAMPLES

### Example 1

### Luciferase Amplicon Expression Vector

Amplicon Production

2618 bp amplicon expression vectors containing an hCMV promoter, firefly luciferase open reading frame, and bGH terminator were synthesized via PCR amplification. Four different versions of luciferase amplicon expression vectors were synthesized as follows: two amplicons produced with MyFi DNA polymerase (Bioline), a relatively high fidelity proofreading polymerase, one with and one without phosphorothioate-modification, and 2 amplicons produced with Biolase DNA polymerase (Bioline) a lower fidelity non-proof reading polymerase, one with and one without phosphorothioate-modification.

For the two luciferase amplicon expression vectors produced via MyFi DNA polymerase, each 10 ml PCR reaction utilized the following reagents: 2 mL of 5× buffer, 0.5 mL of MyFi DNA polymerase, 50 μL of each forward and reverse primer (100 μM), 500 ng of template, PCR water QS to 10 mL. PCR amplification was performed using the following program: initial denature at 95° C. for 60 second, then 30 cycles of two-step PCR of 94° C. for 20 second, 70° C. for 3 minutes; final extension at 70° C. for 5 minutes. The final yield was 50 mg/L.

For the two luciferase amplicon expression vectors produced via Biolase polymerase, each 10 ml PCR utilized the following reagents: 1 mL of 10× buffer, 0.5 mL of MgCl2(50 mM), 0.325 mL of dNTP (40 mM), 1 mL of Biolase, 50 μL of each forward and reverse primer (100 μM), 500 ng of template, 1.25 mL of Betaine(4M), PCR water QS to 10 mL. PCR amplification was performed using the following program: Initial denature at 95° C. for 60 seconds, then 30 cycles of two-step PCR of 94° C. for 20 seconds, 70° C. for 6 minutes; final extension at 72° C. for 5 minutes. The final yield was 50 mg/L.

For all amplicon expression vectors produced via PCR, yield and size was confirmed by Agilent Bioanalyzer 2100. All amplicon expression vectors where purified via ethanol precipitation and anion column exchange to remove unincorporated primers, dNTP and small DNA fragments.
In Vitro Expression Test

Following production of the luciferase amplicon expression vectors, Hela cells were plated in a 96 well plate at 1×10⁴ cells per well. Equal numbers of different Hela cells were transfected with equimolar concentrations of the template luciferase plasmid expression vector and each of the four luciferase amplicon expression vectors (1-0.3-0.1-0.003-0.01 μg). Lipofectamine 2000 (Thermo Fisher) was used for chemical transfection according to the manufacturer's instructions. After a period of 24 h, luciferin was added to the 96 well plate and the resulting light emission measured via a Xenogen IVIS 200 Imaging Instrument (Perkin Elmer).

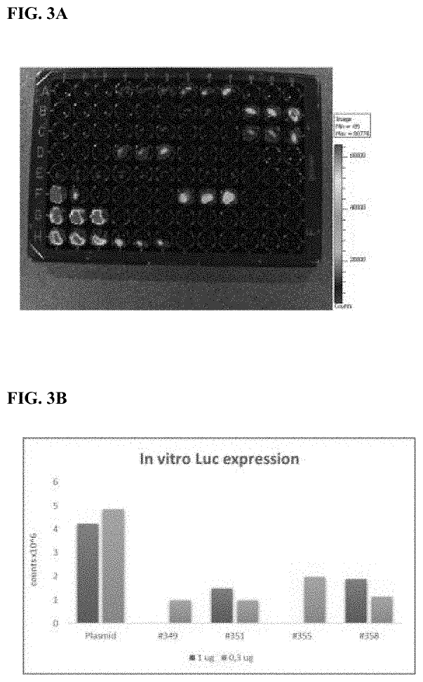

As shown on FIGS. 3A and 3B, all luciferase amplicon expression vectors showed luciferase expression and enzymatic activity in vitro 24 h post transfection.
In Vivo Expression Test

BALB/c mice (5 mice/group, for a total of 65 mice+controls) were anaesthetized using 97% oxygen and 3% isoflurane (Isoba, MSD Animal Health, Walton, UK) then injected by DNA electroporation with equimolar concentrations of luciferase plasmid expression vector (50-10-1 μg/mouse) and one of the four luciferase amplicon expression vectors (18,75-3,75-0,38 μg/mouse) in a 50 μg volume in tibialis muscle. The mice were electroporated by means of a Vet-ePorator Device EPS01 (Evvivax, Italy) and plate electrodes with the following electrical conditions in Electro-Gene-Transfer (EGT) modality: 8 pulses 20 msec each at 110V, 8 Hz, 120 msec interval. Post electroporation, imaging of the electroporated mice was performed under gas anesthesia with a Xenogen IVIS 200 at 24 h, 48 h and 1-week time points.

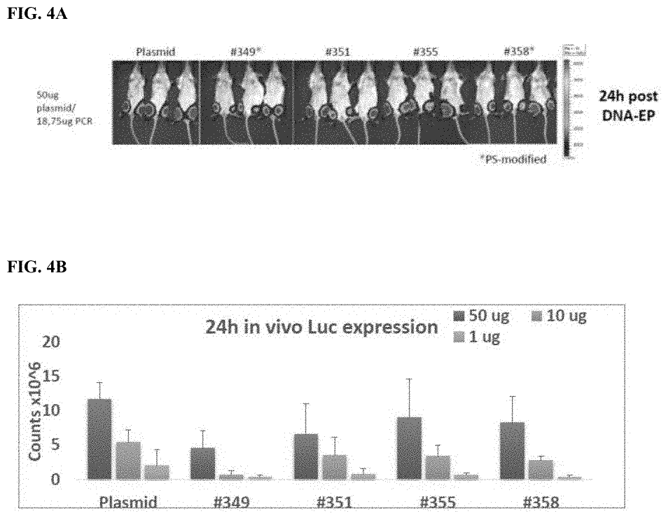

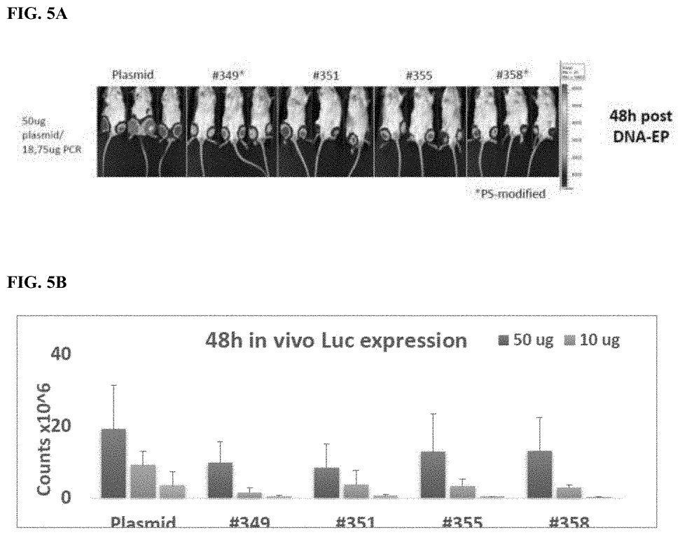

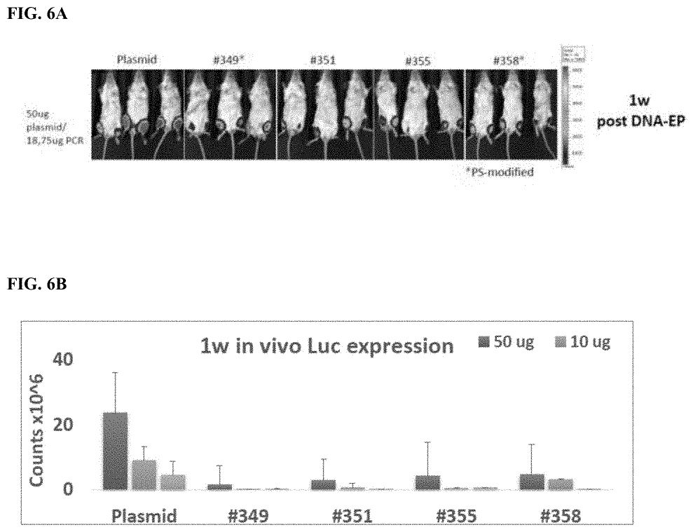

Luciferase expression in vivo of amplicon expression vectors at 24 h post electroporation is shown in FIGS. 4A and 4B. Luciferase expression in vivo of amplicon expression vectors at 48 h post electroporation is shown in FIGS. 5A and 5B. Luciferase expression of amplicon expression vectors in vivo at 1 week post electroporation is shown in FIGS. 6A and 6B.

### Example 2

### dTERT Amplicon Expression Vector

Amplicon Production

4446 bp amplicon expression vectors comprising an hCMV promoter, an open reading frame for a codon optimized canine telomerase reverse transcriptase (dTERT), heat-labile toxin B subunit of E. coli (LTB) and a bGH terminator were synthesized via PCR from a template. Four different versions of dTERT amplicon expression vectors were synthesized as follows: two amplicons produced with Ranger DNA polymerase (Bioline), a high fidelity proofreading polymerase, one with and one without phosphorothioate-modification, and two amplicons produced with Biolase DNA polymerase (Bioline) a lower fidelity non-proof reading polymerase, one with and one without phosphorothioate-modification termini modification.

For the dTERT amplicon expression vectors produced via Ranger DNA polymerase, each 10 ml PCR reaction utilized the following reagents: 2 mL of 5× buffer, 0.1 mL of Ranger DNA polymerase, 50 μL of each forward and reverse primer (100 μM), 500 ng of template, PCR water QS to 10 mL. PCR was performed using the following program: Initial denature at 95° C. for 60 seconds, then 28˜30 cycles of two-step PCR of 95° C. for 20 seconds, 70° C. for 3 minutes; final extension at 70° C. for 5 minutes. The final yield was 50 mg/L.

For the dTERT amplicon expression vectors produced via Biolase DNA polymerase, each 10 ml PCR reaction utilized the following reagents: 1 mL of 10× buffer, 0.4 mL of MgCl2(50 mM), 0.25 mL of dNTP (40 mM), 0.4 mL of Biolase, 50 μL of each forward and reverse primer (100 μM), 500 ng of template, 2.5 mL of Betaine(4M), PCR water QS to 10 mL. PCR was performed using the following program: Initial denature at 95° C. for 60 seconds, then 28˜30 cycles of two-step PCR of 94° C. for 20 second, 70° C. for 6 minutes; final extension at 72° C. for 8 minutes. The final yield was 50 mg/L.

For all dTERT amplicon expression vectors, yield and size was confirmed by Agilent Bioanalyzer 2100. All dTERT amplicon expression vectors where purified via ethanol precipitation and anion column exchange to remove unincorporated primers, dNTP and small DNA fragments.
In Vivo Immunogenicity of dTERT Amplicon Vectors

BALB/c mice (5 mice/group, for a total of 65 mice+controls) were anesthetized using 97% oxygen and 3% isoflurane (Isoba, MSD animal Health, Walton, UK) then injected by DNA electroporation with equimolar concentrations of dTERT DNA plasmid (50-10-5 μg/mouse) or one of the four dTERT amplicon expression vectors (18,75-3,75-1,87 μg/mouse) in a 50 μl volume in tibialis muscle. Mice were electroporated by means of a Vet-ePorator Device EPS01 (Evvivax, Italy) and plate electrodes with the following electrical conditions in Electro-Gene-Transfer (EGT) modality: 8 pulses 20 msec each at 110V, 8 Hz, 120 msec interval.

Mice received two electroporated injections at day 0 and at day 21. The immune response was measured by Intracellular staining on PBMCs at day 28. After prime-boost immunization, immune response against canine telomerase for the sample mouse population was assessed by intracellular staining (ICS) for cytokines by means of a 13 color flow-cytometer (Cytoflex, Beckman Coulter) after O/N stimulation with a pool of 15 mer peptides overlapping by 11 residues and covering the immune dominant portion of canine telomerase for BALB/c haplotype (H-2d).

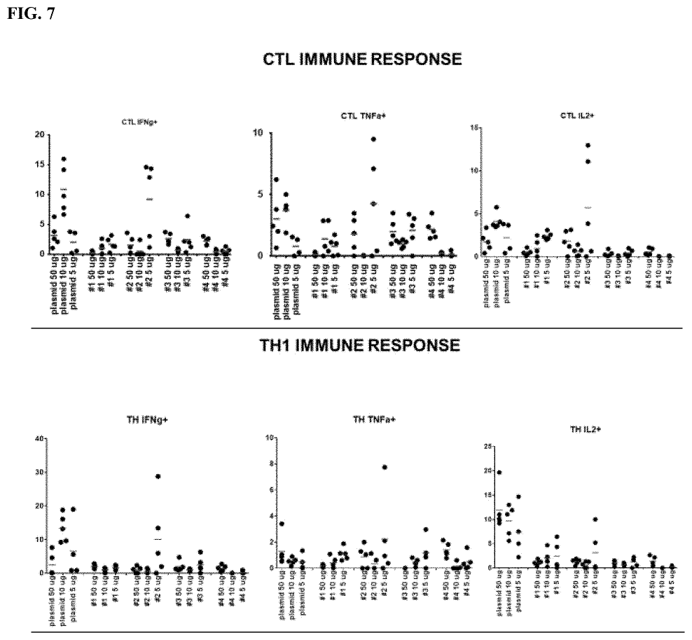

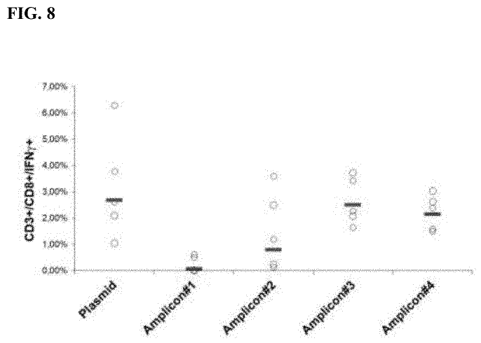

As shown in FIG. 7, the ICS analysis revealed a detectable multicytokine, CD8+ and CD4+ specific immune response in all groups treated with the dTERT amplicon expression vectors. CD8+/IFN [symbol] + specific data was extrapolated for ease of analysis and is shown in FIG. 8. In vivo immunogenicity was observed with all dTERT amplicon expression vectors.

### Example 3

### TERT Consensus Sequence Amplicon Expression Vector Antitumoral Effects

An amplicon expression vector as shown in FIG. 9B was manufactured via PCR utilizing Q5 high-fidelity polymerase (NEB Biolabs, USA) and phosphorothioate modified primers to create a large number of amplicon expression vectors encoding a consensus sequence of TERT as well as TPA and an immunomodulator. A hCMV promoter and bGH terminator were utilized. The resultant 6486 bp amplicons were filtered through a Corning 100 k molecular weight cut-off filter and concentrated to an efficacious dose level via ethanol precipitation.

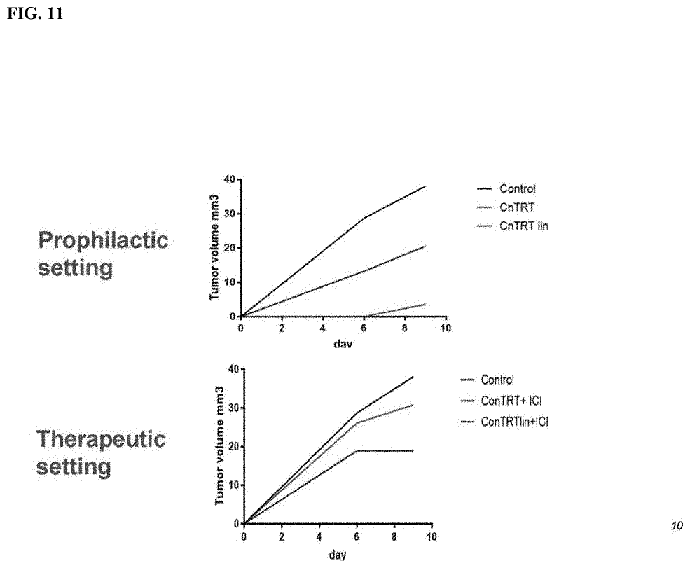

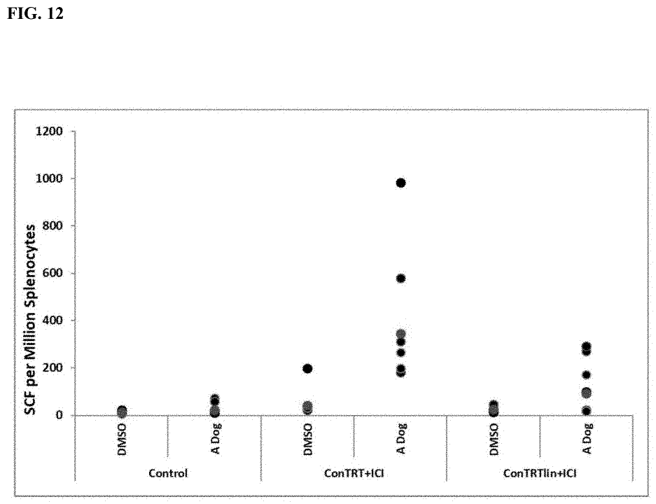

The purified and concentrated amplicon expression vectors where then administered to CT26 tumor model mice via electroporation in both a prophylactic setting and a therapeutic setting. As shown in FIG. 11, in the prophylactic setting the amplicon expression vector showed strong antitumoral effect. As also shown in FIG. 11, a strong antitumoral effect was likewise observed in the therapeutic setting when the amplicon expression vectors were administered in conjunction with ICIs comprising PD-1 and CTLA-4. In addition, as shown in FIG. 12, immunogenicity was also studied.

### Example 4

### Neoantigen Amplicon Expression Vector Antitumoral Effects

An amplicon expression vector as shown in FIG. 10 was manufactured via PCR utilizing Q5 high-fidelity polymerase (NEB Biolabs, USA) and phosphorothioate modified primers to create a large number of amplicon expression vectors encoding target neoantigens identified in a MC38 colon carcinoma mouse tumor model as well as TPA. A hCMV promoter and bGH terminator were utilized. The resultant 3486 bp amplicons were filtered through a Corning 50 K/100 k molecular weight cut-off filter and concentrated to an efficacious dose level via ethanol precipitation.

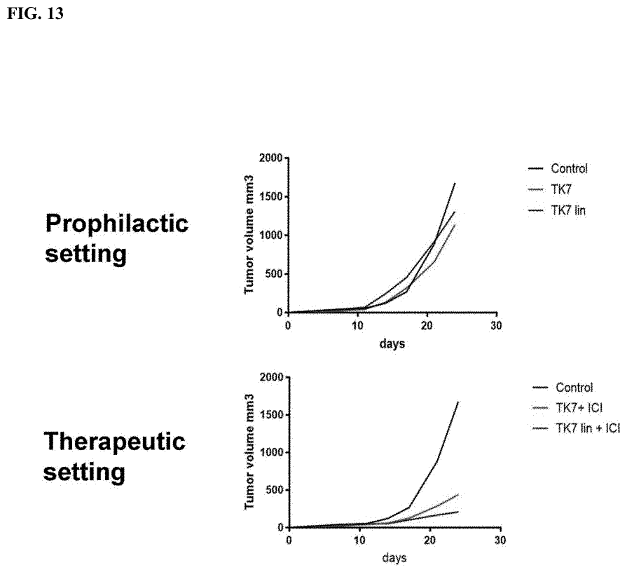

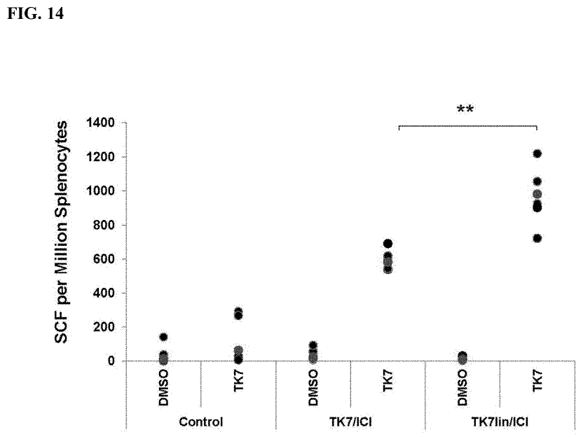

The purified and concentrated amplicon expression vectors where then administered to MC38 colon carcinoma tumor model mice via electroporation in both a prophylactic setting and a therapeutic setting. As shown in FIG. 13, in the prophylactic setting the amplicon expression vector demonstrated moderate antitumoral effect. As also shown in FIG. 13, a very strong antitumoral effect was observed in the therapeutic setting when the amplicon expression vector was administered in conjunction with ICIs comprising PD-1 and CTLA-4. In addition, as shown in FIG. 14, immunogenicity was also studied, showing that the amplicon expression vector, when coupled with ICIs consisting of PD-1 and CTLA-4, showed higher immunogenicity than the same expression vector in plasmid form when also coupled with PD-1 and CTLA-4.

Although the invention has been described with reference to the above examples and embodiments, it is not intended that such references be constructed as limitations upon the scope of this invention except as set forth in the following claims.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention.

What is claimed is:

1. A method of treating cancer in a subject, said method comprising:
Identifying in a subject cancer neoantigens;
Without the use of plasmid-derived DNA, assembling an amplicon expression vector template comprising a promotor, one or more open reading frames (ORF) encoding the identified neoantigens and a terminator via gene synthesis;
amplifying the amplicon expression vector template via the polymerase chain reaction (PCR) and phosphorothioate modified PCR primers to produce a plurality of amplicon expression vectors;
purifying and concentrating the amplicon expression vectors to create an efficacious therapeutic dose for the subject; and
administering the therapeutic dose of amplicon expression vectors via electroporation in conjunction with one or more immune checkpoint inhibitors.

2. The method of claim 1, wherein the immune checkpoint inhibitors include one or more from the group of PD-1, CTLA-4, PD-L1.

3. The method of claim 1, wherein the amplicon expression vector template further comprises an ORF for a small ubiquitin-related modifier (SUMO).

4. The method of claim 1, wherein the amplicon expression vector template further comprises an ORF encoding for tissue plasminogen activator (TPA).

5. The method of claim 2, wherein the amplification via PCR is conducted using a polymerase with an error rate less than 7×10⁻⁵ error/bp.

6. The method of claim 1, wherein the amplicon expression vector template encodes for between 2 and 20 neoantigens.

7. The method of claim 1, wherein the amplicon expression vector template encodes for between 5 and 15 neoantigens.

8. The method of claim 2, wherein the neoantigens are identified from the subject's circulating tumor cells (CTC).

9. The method of claim 4, wherein the amplicon expression vector template further comprises an ORF encoding for an immunomodulator.

10. A method of prophylactically treating cancer in a subject, said method comprising:
Without the use of plasmid-derived DNA, assembling an amplicon expression vector template comprising a promotor, an ORF encoding a consensus sequence for telomerase reverse transcriptase and terminator;
amplifying the amplicon expression vector template via the polymerase chain reaction (PCR) and phosphorothioate modified PCR primers to produce a plurality of amplicon expression vectors;
purifying and concentrating the amplicon expression vectors to create an efficacious prophylactic dose for the subject; and
administering the prophylactic dose of amplicon expression vectors via electroporation.

11. The method of claim 10, wherein the amplification via PCR is conducted using a polymerase with an error rate less than 7×10⁻⁵ error/bp.

12. The method of claim 11, wherein the amplicon expression vector template further comprises an ORF for a small ubiquitin-related modifier (SUMO).

13. The method of claim 10, wherein the amplicon expression vector template further comprises an ORF encoding for tissue plasminogen activator (TPA).

14. The method of claim 11, wherein the amplicon expression vector template further comprises an ORF encoding for an immunomodulator.

15. A method of treating cancer in a subject, said method comprising:
Without the use of plasmid-derived DNA, assembling an amplicon expression vector template comprising a promotor, an ORF encoding a consensus sequence for telomerase reverse transcriptase and terminator;
amplifying the amplicon expression vector template via the polymerase chain reaction (PCR) and phosphorothioate modified PCR primers to produce a plurality of amplicon expression vectors;
purifying and concentrating the amplicon expression vectors to create an efficacious therapeutic dose for the subject; and
administering the therapeutic dose of amplicon expression vectors via electroporation in conjunction with one or more immune checkpoint inhibitors.

16. The method of claim 15, wherein the immune checkpoint inhibitors include one or more from the group of PD-1, CTLA-4, PD-L1.

17. The method of claim 16 wherein the amplicon expression vector template further comprises an ORF for a small ubiquitin-related modifier (SUMO).

18. The method of claim 15, wherein the amplicon expression vector template further comprises an ORF encoding for tissue plasminogen activator (TPA).

19. The method of claim 15, wherein the amplicon expression vector template further comprises an ORF encoding for an immunomodulator.

20. The method of claim 15, wherein the amplification via PCR is conducted using a polymerase with an error rate less than 7×10⁻⁵ error/bp.
